(19) United States
(12) Patent Application Publication
(10) Pub. No.: US 2026/0308464 A1
(43) Pub. Date: Oct. 8, 2026

(54) SYSTEMS AND PROCESSES FOR PRODUCING LINEAR ALPHA OLEFINS FROM FATTY ACIDS UTILIZING A SUPERCRITICAL WATER UNIT

(71) Applicant: Saudi Arabian Oil Company, Dhahran (SA)

(72) Inventors: Ki-Hyouk Choi, Dhahran (SA); Jumanah Abdulkarim Alsaeed, Dhahran (SA)

(73) Assignee: Saudi Arabian Oil Company, Dhahran (SA)

(21) Appl. No.: 19/171,826

(22) Filed: Apr. 7, 2025

(51) Int. Cl. C10G 69/02; B01J 3/00

(52) U.S. Cl. CPC C10G 69/02; B01J 3/008; C10G 2300/1007; C10G 2300/4006; C10G 2300/4012; C10G 2300/4018; C10G 2300/70; C10G 2400/22

(57) ABSTRACT

A process for producing linear alpha olefins utilizing a supercritical water unit may comprise phenylating a free fatty acid stream with an aromatics stream in the presence of a Lewis Acid catalyst, thereby forming a phenylated free fatty acid stream; deoxygenating the phenylated free fatty acid stream to form an alkylaromatic stream; introducing water and the alkylaromatic stream to a supercritical water unit, wherein the water is either at supercritical conditions prior to entering the supercritical water unit or converted to supercritical conditions within the supercritical water unit; and converting the alkylaromatic stream with the water within the supercritical water unit, thereby forming a linear alpha olefin-containing product.

### FIELD

The present disclosure relates to the field of hydrocarbon conversion. More particularly, it relates to systems and methods of converting hydrocarbons to linear alpha olefins (LAO) utilizing a supercritical water unit, as detailed herein.

### BACKGROUND

Olefins, including α-olefins (Alpha-olefins), are useful and valuable chemicals when used as a raw material. For example, α-olefins can be used as a raw material for the production of linear low density polyethylene (LLDPE), high density polyethylene (HDPE), polyalphaolefin (PAO), linear alkyl benzene (LAB), and linear alkyl benzene sulfonate (LABS).

Alpha-olefins for use as a raw material are generally produced as the primary production product, such as in the Ziegler process, which utilizes organometallic catalyst and oligomerization or in catalytic dehydrogenation of n-Paraffins, such as in UOP's Pacol process. However, catalytic processes are very expensive and utilize limited feedstocks such as ethylene.

Non-catalytic processes for wider ranges of feedstocks include thermal cracking of residue or crude oils. Thermal cracking methods generally subject C₂₀-C₃₀ range hydrocarbons, including waxes, to elevated temperatures in the presence of steam, which results in cracking. Conversion in thermal cracking processes is about 25% and the product is 90-95% α-olefins. However, thermal cracking products with wax as feed contain a wide range α-olefins, only some of which are useful in polyalphaolefin production. For crude oils feeds, desired alpha olefin fractions are not readily or economically separable from other components such as n-paraffins and aromatics, such that further thermal decomposition of the alpha olefin compounds results in formation of aromatics through cyclization and alpha olefins compounds' inherent instability.

Accordingly, methods are desired by which alpha olefins, and particularly linear alpha olefins, may be selectively produced from hydrocarbon feedstocks. Supercritical water is one such pathway that has been investigated to produce alpha-olefins from various feedstocks. For example, supercritical water is known to have unique properties for upgrading, desulfurization, and demetallization, among other processes.

### SUMMARY

When hydrocarbons are exposed to supercritical water, alkyl radicals formed under thermal decomposition, including alkyl aromatic radicals, can undergo propagation by two paths: hydrogen abstraction and beta-scission. These propagation pathways are moderated by the supercritical water acting as a diluent, creating a “cage effect” that surrounds local groups of radicals and suppresses inter-radical and intermolecular reactions.

Hydrogen abstraction takes hydrogen from other compounds, with the alkyl radicals subsequently being converted to alkanes. However, hydrogen abstraction reactions are suppressed in supercritical water reactions due to the dilution effect, making it difficult to find hydrogen donor compounds and thereby only a minor amount of alpha olefin production occurs by this pathway. Positively however, this dilution effect also suppresses isomerization from α-olefins to inner olefins (where the double bond is not at the alpha position).

In beta-scission, the alkyl radical cracks to produce an alkyl radical and alpha-olefins. Beta-scission does not require additional molecules. Accordingly, due to not requiring donor compounds, beta-scission of alkyl radicals is increased under supercritical water conditions. Thus, under supercritical water conditions, more α-olefins tend to be formed than under conventional thermal cracking conditions, primarily under the beta scission pathway.

Consequently, the primarily beta-scission reactions of supercritical water with hydrocarbons provides the opportunity to provide tailored feedstocks to supercritical water conversion that will directly form linear alpha olefins as the primary product of the initial beta-scission reaction pathway.

Accordingly, embodiments herein provide such tailored feedstocks for linear alpha olefin production by reacting free fatty acids and aromatics in phenylation and deoxygenation (decarboxylation/decarbonylation) reactions to form an alkylaromatic feed from which the primary beta scission products are at least one (and often two) linear alpha olefin and an aromatic compound, as explained in further detail herein.

In accordance with one embodiment of the present disclosure, a process for producing linear alpha olefins utilizing a supercritical water unit may comprise phenylating a free fatty acid stream with an aromatics stream in the presence of a Lewis Acid catalyst, thereby forming a phenylated free fatty acid stream; deoxygenating the phenylated free fatty acid stream to form an alkylaromatic stream; introducing water and the alkylaromatic stream to a supercritical water unit, wherein the water is either at supercritical conditions prior to entering the supercritical water unit or converted to supercritical conditions within the supercritical water unit; and converting the alkylaromatic stream with the water within the supercritical water unit, thereby forming a linear alpha olefin-containing product.

In accordance with another embodiment of the present disclosure, a system for producing linear alpha olefins may comprise a pretreatment unit comprising a phenylation unit and a deoxygenation unit; and a supercritical water unit fluidly connected downstream from the deoxygenation unit of the pretreatment unit.

In at least the previous embodiment, the phenylation unit may be configured to receive a free fatty acid stream and an aromatics stream, the deoxygenation unit may be fluidly connected downstream from the phenylation unit, and the deoxygenation unit may be configured to receive at least a phenylated free fatty acid stream. Further, the supercritical water unit may be configured to receive an alkylaromatic stream from the deoxygenation unit and water, operate under supercritical water conditions, and to produce a linear alpha olefin-containing product.

Additional features and advantages of the described embodiments will be set forth in the detailed description which follows, and in part will be readily apparent to those skilled in the art from that description or recognized by practicing the described embodiments, including the detailed description which follows as well as the claims.

### BRIEF DESCRIPTION OF THE DRAWINGS

The following detailed description of specific embodiments of the present disclosure can be best understood when read in conjunction with the following drawings, where like structure is indicated with like reference numerals and in which:

FIG. 1 illustrates one or more reactions for converting free fatty acids and aromatics to linear alpha olefins, according to embodiments herein; and

FIG. 2 illustrates a system for producing linear alpha olefins utilizing a supercritical water unit, according to embodiments herein.

For the purpose of describing the simplified schematic illustrations and descriptions of the relevant figures, the numerous valves, temperature sensors, electronic controllers and the like that may be employed and well known to those of ordinary skill in the art of certain chemical processing operations are not included. It should be understood that these components are within the spirit and scope of the present embodiments disclosed. However, operational components, such as those described in the present disclosure, may be added to the embodiments described in this disclosure.

It should further be noted that arrows in the drawings refer to process streams. However, the arrows may equivalently refer to transfer lines, which may serve to transfer process streams between two or more system components. Additionally, arrows that connect to system components define inlets or outlets in each given system component. The arrow direction corresponds generally with the major direction of movement of the materials of the stream contained within the physical transfer line signified by the arrow. Furthermore, arrows, which do not connect two or more system components, signify a product stream, which exits the depicted system, or a system inlet stream, which enters the depicted system. Product streams may be further processed in accompanying chemical processing systems or may be commercialized as end products. System inlet streams may be streams transferred from accompanying chemical processing systems or may be non-processed feedstock streams. Some arrows may represent recycle streams, which are effluent streams of system components that are recycled back into the system. However, it should be understood that any represented recycle stream, in some embodiments, may be replaced by a system inlet stream of the same material, and that a portion of a recycle stream may exit the system as a product.

Additionally, arrows in the drawings may schematically depict process steps of transporting a stream from one system component to another system component. For example, an arrow from one system component pointing to another system component may represent “passing” a system component effluent to another system component, which may include the contents of a process stream “exiting” or being “removed” from one system component and “introducing” the contents of that product stream to another system component.

It should be understood that according to the embodiments presented in the relevant figures, an arrow between two system components may signify that the stream is not processed between the two system components. In other embodiments, the stream signified by the arrow may have substantially the same composition throughout its transport between the two system components. Additionally, it should be understood that in embodiments, an arrow may represent that at least 75 wt. %, at least 90 wt. %, at least 95 wt. %, at least 99 wt. %, at least 99.9 wt. %, or even 100 wt. % of the stream is transported between the system components. As such, in embodiments, less than all of the stream signified by an arrow may be transported between the system components, such as if a slip stream is present.

It should be understood that two or more process streams are “mixed” or “combined” when two or more lines intersect in the schematic flow diagrams of the relevant figures. Mixing or combining may also include mixing by directly introducing both streams into a like reactor, separation unit, or other system component. For example, it should be understood that when two streams are depicted as being combined directly prior to entering a separation unit or reactor, that in embodiments the streams could equivalently be introduced into the separation unit or reactor and be mixed in the reactor. Alternatively, when two streams are depicted to independently enter a system component, they may in embodiments be mixed together before entering that system component.

Reference will now be made in greater detail to various embodiments, some embodiments of which are illustrated in the accompanying drawings. Whenever possible, the same reference numerals will be used throughout the drawings to refer to the same or similar parts.

### DETAILED DESCRIPTION

As previously stated, embodiments described herein generally relate to the field of hydrocarbon conversion and to systems and methods of converting hydrocarbons to linear alpha olefins utilizing a supercritical water unit.

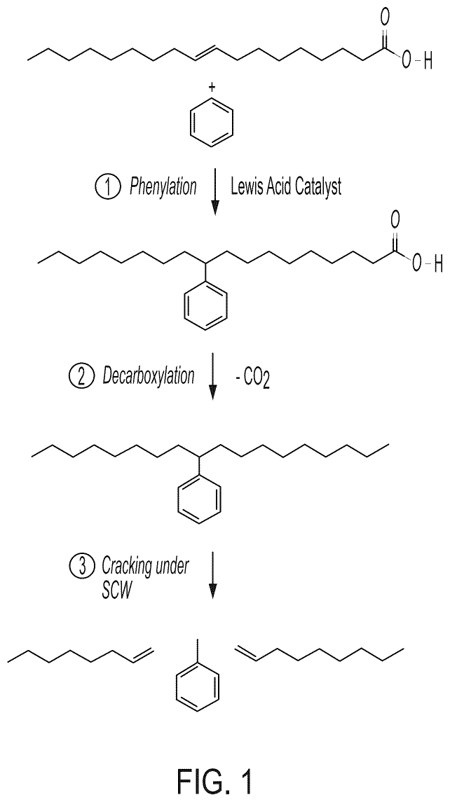

Particularly, and now referring to FIG. 1, free fatty acids (such as but not limited to oleic acid as shown) may be exposed to an aromatics stream (such as but not limited to benzene as shown) in the presence of a Lewis Acid catalyst, which may prompt reaction of the aromatic compound with free fatty acid. The free fatty acid may then be phenylated with the aromatic compound, forming a phenylated free fatty acid (such as but not limited to 10-Phenyloctadecanoic acid as shown).

Still referring to FIG. 1, the phenylated free fatty acid may then be deoxygenated by removal of the carbonyl, carboxyl groups, and/or oxygen groups to form an alkylaromatic. Further, when subsequently subjected to supercritical water, beta scission may break the phenyl group bond between the deoxygenated alkyl group and the aromatic, essentially forming a phenyl and one or two linear hydrocarbon chains. These linear hydrocarbon chains, aided by the caging effect of supercritical water, may subsequently form linear olefins with double bonds at the alpha position.

As used in this disclosure, a “separation unit” refers to any separation device that at least partially separates one or more chemicals that are mixed in a process stream from one another. For example, a separation unit may selectively separate differing chemical species, phases, or sized material from one another, forming one or more chemical fractions. Examples of separation units include, without limitation, distillation columns, flash drums, knock-out drums, knock-out pots, centrifuges, cyclones, filtration devices, traps, scrubbers, expansion devices, membranes, solvent extraction devices, and the like. It should be understood that separation processes described in this disclosure may not completely separate all of one chemical constituent from all of another chemical constituent. It should be understood that the separation processes described in this disclosure “at least partially” separate different chemical components from one another, and that even if not explicitly stated, it should be understood that separation may include only partial separation. As used in this disclosure, one or more chemical constituents may be “separated” from a process stream to form a new process stream. Generally, a process stream may enter a separation unit and be divided, or separated, into two or more process streams of desired composition. It should be understood that an “effluent” generally refers to a stream that exits a system component such as a separation unit, a reactor, or reaction zone, following a particular reaction or separation, and generally has a different composition (at least proportionally) than the stream that entered the separation unit, reactor, or reaction zone.

It should further be understood that streams and/or fractions, as the terms are used interchangeably herein, may be named for the components of the stream, and the component for which the stream is named may be the major component of the stream (such as comprising from 50 weight percent (wt. %), from 70 wt. %, from 90 wt. %, from 95 wt. %, from 99 wt. %, from 99.5 wt. %, or even from 99.9 wt. % of the contents of the stream to 100 wt. % of the contents of the stream). It should also be understood that components of a stream are disclosed as passing from one system component to another when a stream comprising that component is disclosed as passing from that system component to another. For example, a disclosed “aromatics stream” passing from a first system component to a second system component should be understood to equivalently disclose “aromatics” passing from a first system component to a second system component, and the like.

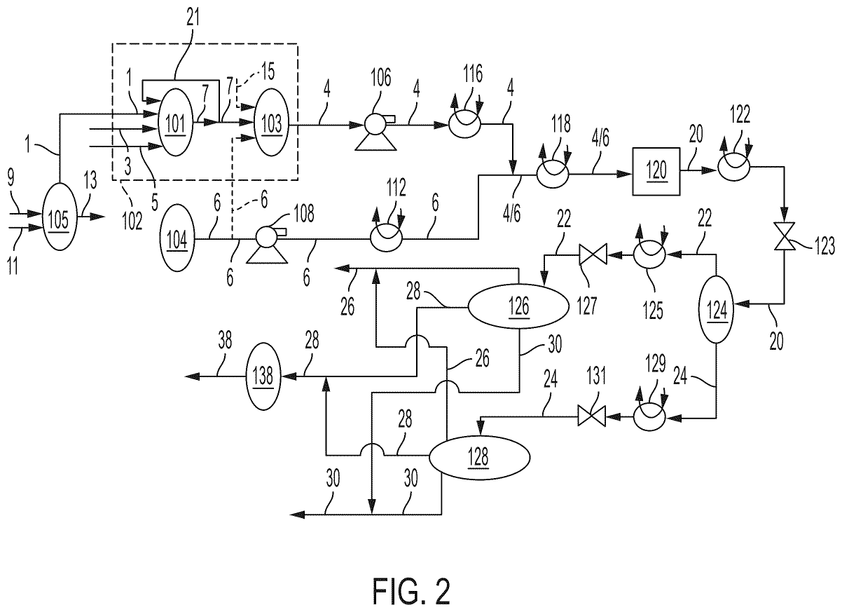

Referring now to FIG. 2, illustrated is a system 100 for converting an alkylaromatic stream 4 in a supercritical water unit 120 with water 6. As shown in FIG. 1, the water 6 may originate from a water storage medium 104, which may be fluidly connected upstream of the supercritical water unit 120. The alkylaromatic stream 4 may originate from a pretreatment unit 102, which may be fluidly connected upstream of the supercritical water unit 120. The pretreatment unit 102 may itself comprise a phenylation unit 101 and a deoxygenation unit 103.

As previously referenced, the phenylation unit 101 may be configured to receive a free fatty acid stream 1, an aromatics stream 3, and a Lewis Acid catalyst 5. The phenylation unit 101, with the assistance of the Lewis Acid catalyst 5, may then react the aromatics in the aromatics stream 3 and the free fatty acid stream 1 to produce phenylated fatty acid. Accordingly, the phenylation unit 101 may form a phenylated free fatty acid stream 7. The free fatty acid stream may comprise palmitic acid, stearic acid, oleic acid, linoleic acid, arachidic acid, eicosenoic acid, behenic acid, or combinations thereof, although other C₁₂-C₂₀ free fatty acids are contemplated. The aromatics stream 3 may generally include BTX (benzene, toluene, xylene), although other aromatics such as ethylbenzene and diethylbenzene are also contemplated. The Lewis Acid catalyst 5 is not particularly limited, and may include, but may not be limited to, AlCl₃ BF₃, an acidic zeolite, or combinations thereof.

The phenylation unit 101 may operate at, and thereby the phenylation may occur at, an operating temperature of from 10° C. to 500° C. (such as from 30° C. to 400° C.); an operating pressure of from 0.1 megapascals (MPa) to 15 MPa (such as from 0.1 MPa to 10 MPa); a weight hourly space velocity (WHSV) of from 0.05 hr⁻¹ to 100 hr⁻¹ (such as from 0.1 hr⁻¹ to 10 hr⁻¹); or combinations thereof. A ratio of the free fatty acid stream 1 to the aromatics stream 3 sent to the phenylation unit 101 may also be from 1:2 to 1:100. The phenylation unit may be a fixed catalyst bed reactor, a fluidized catalyst bed reactor, an ebullated catalyst bed reactor, a slurry catalyst bed reactor, or combinations thereof.

Without being limited by theory, free fatty acids are generally regarded as a waste product associated with used cooking oils, i.e., triglycerides. Accordingly, in embodiments, the free fatty acid stream 1 may include a variety of free fatty acids, such as saturated and unsaturated free fatty acids, including but not limited to those derived from triglycerides such as vegetable oils or animal fats. Accordingly, in at least some embodiments, the system 100 may comprise a free fatty acid generation unit 105 which may be configured to receive a triglyceride stream 9 and a second water stream 11. The triglyceride stream 9 may include waste vegetable oils or waste animal fats, which may include at least a fouled portion (free fatty acids) and unfouled portion. The free fatty acid generation unit 105 may subsequently be configured to hydrolyze the triglyceride stream 9 in the presence of the second water stream 11 to form the free fatty acid stream 1 and glycerol 13, such as but not limited to by the Colgate-Emery process. The free fatty acid generation unit 105 may also include a separation component configured to separate the free fatty acid stream 1 from the glycerol 13, water, and other byproducts.

Further, in at least some embodiments, the aromatics stream 3 may be sourced from an upstream refining unit, such as but not limited to a catalytic reformer, a steam cracker, an aromatization unit, a coal extraction plant, or similar.

Still referring to FIG. 2, the phenylated free fatty acid stream 7 may subsequently be received by the deoxygenation unit 103, which may be fluidly connected downstream from the phenylation unit 101 and upstream from the supercritical water unit 120. The deoxygenation unit 103 may in turn be configured to deoxygenate the phenylated free fatty acid stream 7, such as by removing carboxyl groups, carbonyl groups, oxygen from the same, such that the alkylaromatic stream 4 and more specifically alkylaromatics are formed. In embodiments, the deoxygenation unit 103 may be configured to deoxygenate the phenylated free fatty acid stream 7 via thermal cracking, catalytic cracking, or hydrogenation, which may be carried out with one or more co-feeds, such as a hydrogen stream 15 for the hydrogenation.

For example, and in embodiments, the deoxygenation unit 103 may be configured to additionally receive water 6, steam, or both, such that the thermal cracking resembles steam cracking. Additionally, the deoxygenation unit 103 may be configured to receive and/or house one or more catalysts, such that the catalytic cracking resembles steam enhanced catalytic cracking or the hydrogenation resembles catalytic hydrogenation. In embodiments, the hydrogenation catalyst may comprise a noble metal catalyst, a transition metal sulfide catalyst, or both. Further, in the event the deoxygenation is hydrogenation, the hydrogen stream 15 may include a co-feed of inert gas, such as but not limited to nitrogen or a noble gas.

In at least some embodiments, the deoxygenation unit 103 may also be configured to receive an n-alkane solvent stream, such that the phenylated free fatty acid stream 7 is deoxygenated in the presence of the n-alkane solvent. In embodiments, the n-alkane solvent may include n-alkanes having greater than or equal to six carbons, such as from C₆ alkanes to C₃₀ alkanes, for example. Without being limited by theory, the n-alkane solvent may be utilized as a diluent to reduce further reactions of the deoxygenated phenylated free fatty acid stream, due to the deoxygenation reaction generally be exothermic. Similar to the free fatty acid generation unit 105 and the phenylation unit 101, the deoxygenation unit 103 may also include a separation component configured to separate the alkylaromatic stream 4 from the water 6, steam, hydrogen, or n-alkane solvent, as appropriate.

In the event the deoxygenation unit 103 is configured for hydrogenation, the deoxygenation may occur at a temperature of greater than 250° C., such as from 250° C. to 300° C., from 300° C. to 350° C., from 350° C. to 400° C., from 400° C. to 450° C., from 450° C. to 500° C., or combinations of the previous ranges or smaller ranges therein, such as from 250° C. to 350° C., or from 250° C. to 500° C. Similarly, the deoxygenation may occur at a pressure of from greater than 0.5 MPa, such as from 0.5 MPa to 1 MPa, from 1 MPa to 1.5 MPa, from 1.5 MPa to 2 MPa, from 2 MPa to 4 MPa, from 4 MPa to 6 MPa, from 6 MPa to 8 MPa, from 8 MPa to 10 MPa, or combinations of the previous ranges or smaller ranges therein, such as from 0.5 MPa to 10 MPa, or from 4 MPa to 8 MPa. Also similarly, the deoxygenation may occur at a liquid hourly space velocity of greater than 0.1 hr⁻¹, such as from 0.1 hr⁻¹ to 0.2 hr⁻¹, from 0.2 hr⁻¹ to 0.4 hr⁻¹, from 0.4 hr⁻¹ to 1 hr⁻¹, from 1 hr⁻¹ to 2 hr⁻¹, from 2 hr⁻¹ to 4 hr⁻¹, from 4 hr⁻¹ to 5 hr⁻¹, or combinations of the previous ranges or smaller ranges therein, such as from 0.1 hr⁻¹ to 5 hr⁻¹, or from 0.4 hr⁻¹ to 4 hr⁻¹.

In the event the deoxygenation unit 103 is configured for thermal or steam cracking, the deoxygenation may occur may occur at a temperature of greater than 300° C., such as from 300° C. to 350° C., from 350° C. to 400° C., from 400° C. to 450° C., from 450° C. to 500° C., from 500° C. to 550° C., from 550° C. to 600° C., or combinations of the previous ranges or smaller ranges therein, such as from 300° C. to 600° C., or from 400° C. to 500° C. Similarly, the deoxygenation may occur at a pressure of from greater than 0.1 MPa, such as from 0.1 MPa to 0.5 MPa, from 0.5 MPa to 1 MPa, from 1 MPa to 1.5 MPa, from 1.5 MPa to 2 MPa, from 2 MPa to 4 MPa, from 4 MPa to 6 MPa, from 6 MPa to 8 MPa, from 8 MPa to 10 MPa, or combinations of the previous ranges or smaller ranges therein, such as from 0.5 MPa to 10 MPa, or from 4 MPa to 8 MPa. Also similarly, the deoxygenation may occur at a residence time of greater than 1 minute, such as from 1 minute to 2 minutes, from 2 minutes to 10 minutes, from 10 minutes to 30 minutes, from 30 minutes to 60 minutes, from 60 minutes to 90 minutes, from 90 minutes to 120 minutes, or combinations of the previous ranges or smaller ranges therein, such as from 1 minute to 120 minutes, or from 10 minutes to 60 minutes.

In at least some embodiments, the deoxygenation unit may be a CSTR reactor, a tubular-type continuous reactor, a vessel-type continuous reactor, or combinations thereof.

As previously stated, the supercritical water unit 120 may be configured to receive the alkylaromatic stream 4 and the water 6. The water 6 may originate from a water storage medium 104. However, the water 6 may also be pretreated such that it meets one or more criteria for supercritical water. For example, and in embodiments, the water 6 may comprise a demineralized water; a conductivity of less than 1 microsiemens per centimeter (μS/cm) (such as from 1 to 0.01 μS/cm; a sodium content of less than 5 micrograms per liter (μg/L) (such as from 0.001 μg/L to 5 μg/L); a chloride content of less than 5 micrograms per liter (such as from 0.001 μg/L to 5 μg/L); a silica content of less than 3 micrograms per liter (such as from 0.001 μg/L to 3 μg/L); or combinations thereof.

As previously stated, the system 100 may comprise the supercritical water unit 120. The supercritical water unit 120 is not particularly limited, and may be a tubular type reactor, a vessel-type reactor, or a combination tubular-vessel-type reactor. Further, the supercritical water unit 120 may be horizontal flow, upflow, or downflow. Further yet, the supercritical water unit 120 may have a residence time of from 10 seconds to 60 minutes, or any smaller range therein such as from 30 seconds to 30 minutes; an operating temperature of from 380° C. to 500° C. (such that the supercritical water unit 120 operates at supercritical water conditions); or both.

Without being limited by theory, the supercritical water unit 120 may operate at a lower temperature than other methods of supercritical water hydrocarbon conversion (e.g., residual oil) owing to the inclusion of the alkylaromatic stream 4. Particularly, the alkylaromatic stream 4 may be more readily susceptible to cracking than other hydrocarbon streams such as paraffins, such that greater temperatures are not required to start the cracking/beta scission of the alkylaromatic stream 4.

As previously stated, the supercritical water unit 120 may receive the water 6 and the alkylaromatic stream 4. In embodiments, the water 6 and/or alkylaromatic stream 4 may be pre-mixed at a feed mixer (such as where the water 6 and the alkylaromatic stream 4 meet in the figures) prior to being introduced to the supercritical water unit 120. The feed mixer may in turn be fluidly connected downstream of the pretreatment unit 102 and the water storage medium 104, as well as upstream of the supercritical water unit 120.

Additionally, the mixture of the water 6 and the alkylaromatic stream 4 may be preheated at a mixture preheater 118. The mixture preheater 118 may preheat the water-alkylaromatic stream mixture 16 to a temperature of from 380° C. to 450° C. to prior to introducing the water-alkylaromatic stream mixture 16 to the supercritical water unit 120. Accordingly, the mixture preheater 118 may be interposed downstream of the feed mixer and upstream of the supercritical water unit 120.

Further, the system 100 may be configured such that the alkylaromatic stream 4 and the water 6 may be introduced at defined ratios either to the feed mixer or supercritical water unit 120. For example, and in embodiments, an alkylaromatic stream to water ratio within the system 100 may be from 1:1 to 1:10 alkylaromatic stream to water, and/or from 1:2 to 1:5 alkylaromatic stream to water.

As previously stated, the water-alkylaromatic stream mixture 16 may be preheated. Additionally or alternatively, the water 6 and alkylaromatic stream 4 may be preheated individually before introduction to the supercritical water unit 120. For example, and in embodiments, the system 100 may further comprise a water preheater 112 and a feed preheater 116. The feed preheater 116 may be fluidly connected downstream of the pretreatment unit 102, as well as upstream of the supercritical water unit 120 and/or the feed mixer. The water preheater 112 may be fluidly connected downstream of the water storage medium 104, as well as upstream of the supercritical water unit 120 and/or the feed mixer. The feed preheater 116 may preheat the alkylaromatic stream 4 to a temperature of from 50° C. to 250° C. (or from 50° C. to 250° C.) prior to being introduced to the supercritical water unit 120 or the feed mixer. Similarly, the water preheater 112 may preheat the water 6 to a temperature of from 374° C. to 800° C. (or from 400° C. to 550° C.) prior to being introduced to the supercritical water unit 120 or the feed mixer. In embodiments, the feed preheater 116 and the water preheater 112 may preheat the respective streams by heat exchange with a heat exchange fluid such as, but not limited to, water or steam.

Still referring to FIG. 1, the water 6 and/or alkylaromatic stream 4 may also be pressurized prior to being introduced to the supercritical water unit 120, the preheaters, or mixers. For example, and in embodiments, the system 100 may comprise a feed pump 106 fluidly connected downstream of the pretreatment unit 102 and upstream of the supercritical water unit 120. Similarly, the system 100 may comprise a water pump 108 fluidly connected downstream of the water storage medium 104 and upstream of the supercritical water unit 120.

Still referring to FIG. 1, and as previously stated, the supercritical water unit 120 may receive the water 6 and the alkylaromatic stream 4 for converting the alkylaromatic stream 4 into a linear alpha olefin-containing product 20. However, the system 100 may further include one or more downstream separation units for separating the linear alpha olefins from the reminder of the product.

For example, and as shown in FIG. 1, the system 100 may further comprise a gas-liquid separator 124 fluidly connected downstream of the supercritical water unit 120. The gas-liquid separator 124 may be configured to separate the linear alpha olefin-containing product 20 into a gas product stream 22 and a liquid product stream 24. In embodiments, the temperature cut point of the gas product stream 22 and the liquid product stream 24 may be from 100° C. to 250° C.

The system 100 may further comprise a second oil-water separator 128 fluidly connected downstream of the supercritical water unit 120 and the gas-liquid separator 124. The second oil-water separator 128 may be configured to separate the liquid product stream 24 into a gas hydrocarbon product 26, a liquid hydrocarbon product 28, and a water residue stream 30.

The system 100 may further comprise a first oil-water separator 126 fluidly connected downstream of the supercritical water unit 120 and the gas-liquid separator 124. The first oil-water separator 126 may be configured to separate the gas product stream 22 into additional gas hydrocarbon product 26, additional liquid hydrocarbon product 28, and additional water residue stream 30 after condensing the stream 22, although at different product stream ratios than the second oil-water separator 128.

The system 100 may then comprise a fractionation unit 138 fluidly connected downstream from the gas-liquid separator 124 and the first oil-water separator 126. The fractionation unit 138 may be configured to separate the liquid hydrocarbon product 28 into multiple streams by boiling point or chemical structure. Thereby, the products of the fractionation unit 138 may be a linear alpha olefin fraction at a temperature cut point of from −6.3° C. to 315° C.; a BTX fraction at a temperature cut point of from 80° C. to 138° C.; an alkylated benzene fraction at a temperature cut point of from 80° C. to 300° C.; and an unreacted alkylaromatics fraction at a temperature cut point of from 350° C. to 450° C. Particularly, for those fractions with overlapping temperature cut points, the fractionation unit may be configured to separate fractions by extraction or adsorption, such as for separating aromatic (BTX, alkylated benzene, and unreacted alkylaromatics fraction) from non-aromatic (LAO) fractions.

As explained in further detail herein, the linear alpha olefins desired for later polyalphaolefin synthesis may generally have a boiling point between 121° C. (1-Octene) and 213° C. (1-Dodecene), such that the liquid hydrocarbon product 28 may be isolated for additional separation of the linear alpha olefins at or around this temperature range.

Still referring to FIG. 2, in at least some embodiments the linear alpha olefin-containing product 20 may be quenched, depressurized or both prior to being sent to the one or more separation units. Accordingly, the system 100 may further comprise a quenching unit 122 and a depressurization unit 123. The quenching unit 122 may be similar or identical to the feed preheater 116 and/or water preheater 112, i.e., the quenching unit may be a heat exchanger configured to cool/quench the linear alpha olefin-containing product 20 to a temperature of from 20° C. to 250° C., such as with a heat exchange fluid such as, but not limited to, water or steam. The depressurization unit 123 may be configured to depressurize the linear alpha olefin-containing product 20 to a pressure of from 0.1 megapascals (MPa) to 5 MPa.

The system may also comprise heat exchangers 125/129 for cooling the gas product stream 22 and the liquid product stream 24 prior to introduction into the first oil-water separator 126 and the oil-liquid separator 128, respectively, such as with a heat exchange fluid such as, but not limited to, water or steam. Similarly, the system may also comprise depressurizers 127/131 for depressurizing the gas product stream 22 and the liquid product stream 24 prior to introduction into the first oil-water separator 126 and the oil-liquid separator 128, respectively.

As previously stated, there may be various heat exchangers, preheaters, and quench units positioned within the system to heat and cool various streams. Accordingly, in at least some embodiments, the feed preheater 116, the water preheater 112, the mixture preheater 118, the quench unit 122, and the heat exchangers 125/129 may be heat integrated, such that hot heat transfer fluid exiting the quenching units and the heat exchangers may eventually be circulated to the preheaters, and vice versa, such that total energy usage of the system is reduced.

As previously stated, embodiments herein may also be directed to processes of producing linear alpha olefins utilizing the supercritical water unit 120. The methods may include any of the systems hereinbefore described.

In at least some embodiments, the process may comprise introducing the alkylaromatic stream 4 and the water 6 to the supercritical water unit 120; and converting the alkylaromatic stream 4 with the water 6 within the supercritical water unit 120, thereby forming a linear alpha olefin-containing product 20. The water 6 in these embodiments may be either at supercritical conditions prior to entering the supercritical water unit 120 or converted to supercritical conditions within the supercritical water unit 120.

The process may also comprise pretreating in the pretreatment unit 102 to thereby form the alkylaromatic stream 4, the pretreatment unit 102 of which may comprise the phenylation unit 101 and the deoxygenation unit 103. Accordingly, in embodiments, the process may comprise phenylating the free fatty acid stream 1 and the aromatics stream 3 in the presence of a Lewis Acid catalyst 5, thereby forming the phenylated free fatty acid stream 7. The process may then comprise deoxygenating the phenylated free fatty acid stream 7 to form the alkylaromatic stream 4.

In embodiments, and as previously stated, the products of the phenylation unit 101 may in some embodiments not result in complete conversion of the free fatty acid stream 1 and the aromatics stream 3 to the phenylated free fatty acid stream 7. Accordingly, the process may comprise separating a non-phenylated portion of the phenylated free fatty acid stream 7 prior to deoxygenating the phenylated free fatty acid stream 7. The non-phenylated portion 21 may then be recycled for additional phenylation in the phenylation unit 101.

As previously stated, the free fatty acids may be derived from triglycerides, such as, but not limited to triglycerides such as vegetable oils, animal fats, or combinations thereof. Accordingly, the process may comprise hydrolyzing the triglycerides as the triglyceride stream 9 in the presence of a second water stream 11 to form the free fatty acid stream 1 and glycerol 13. The process may also comprise separating the free fatty acid stream 1 from the glycerol 13.

As previously stated, the deoxygenation unit 103 may include thermal cracking, catalytic cracking, or hydrogenation functionalities. Accordingly, deoxygenating the phenylated free fatty acid stream 7 to form the alkylaromatic stream 4 may comprise thermally cracking the phenylated free fatty acid stream 7 in the presence of steam or water 6 to form the alkylaromatic stream 4; catalytically converting the phenylated free fatty acid stream 7 in the presence of a catalyst as well as steam or water 6 to form the alkylaromatic stream 4; or hydrogenating the phenylated free fatty acid stream 7 in the presence of the hydrogen stream 15 to form the alkylaromatic stream 4.

As previously stated, the linear alpha olefin-containing product 20 may be separated in one or more units to isolate the linear alpha olefins. Accordingly, the process may further comprise introducing the linear alpha olefin-containing product 20 to the gas-liquid separator 124 to form the gas product stream 22 and the liquid product stream 24. The process may then comprise introducing the gas product stream 22 to the first oil-water separator 126 and the liquid product stream 24 to the second oil-water separator 128 to form the gas hydrocarbon product 26, the liquid hydrocarbon product 28, and the water residue stream 30. The process may then comprise introducing the liquid hydrocarbon product 28 to the fractionation unit 138, thereby forming the linear alpha olefin fraction; the BTX fraction; the alkylated benzene fraction; the unreacted alkylaromatic fraction, or combinations thereof.

Also as previously stated, it may be desired to quench and/or depressurize the linear alpha olefin-containing product 20 prior to separation. Accordingly, the process may further comprise quenching the linear alpha olefin-containing product 20 prior to sending the linear alpha olefin-containing product 20 to the gas-liquid separator 124; depressurizing the linear alpha olefin-containing product 20 prior to sending the linear alpha olefin-containing product 20 to the gas-liquid separator 124; or both.

Also as previously stated, it may be desired to preheat and/or mix the alkylaromatic stream 4 and the water 6 prior to introduction to the supercritical water unit 120. Accordingly, the process may comprise mixing the water 6 and the alkylaromatic stream 4 prior to the water 6 and the alkylaromatic stream 4 being introduced to the supercritical water unit 120; and preheating the water-alkylaromatic stream mixture 16 to a temperature of from 380° C. to 650° C. prior to introducing the water-alkylaromatic stream mixture 16 to the supercritical water unit 120. Alternatively or additionally, the process may comprise preheating the water 6, the alkylaromatic stream 4, or both prior to introduction into the mixer and/or the supercritical water unit 120.

### Examples

A laboratory scale example was conducted to determine the suitability of the systems and feeds described herein for conversion to linear alpha olefins.

### Phenylation

0.376 kilograms of oleic acid (as fatty acid stream), 0.624 kilograms of benzene (as aromatics stream 3), and 0.179 kilograms of AlCl₃ (as lewis acid catalyst 5) were added to a batch reactor (acting as the phenylation unit) having two liters volume. Due to the amounts of oleic acid and benzene used, the molar ratio of feed was 1:6:1. Nitrogen gas was then added to the reactor for maintaining pressure at about 5 bar gauge. The reactor was then heated to about 70° C. concurrently with stirring of the reactants, with the temperature then maintained for approximately 7 hours. The reactor was then cooled to room temperature, with the internal fluid subsequently transferred to a glass flask. Approximately 1.5 liters of 6 M hydrochloric acid was then added to separate the produced organic and aqueous layers. The organic layer was then taken to evaporation to separate the stearic acid (approximately 0.44 kilograms) from the unreacted materials (oleic acid and benzene). The composition of the stearic acid is shown below in Table 1. The Phenylation example was then subsequently repeated to accumulate the amount of stearic acid for later reactions.

TABLE 1
Stearic Acid Composition

| Stearic Acid | | |
|---|---|---|
| Component | Weight(kg) | Weight % |
| 2-Phenylstearic acid | 0 | 0.0% |
| 3-Phenylstearic acid | 0 | 0.0% |
| 4-Phenylstearic acid | 0 | 0.0% |
| 5-Phenylstearic acid | 0 | 0.0% |
| 6-Phenylstearic acid | 0 | 0.0% |
| 7-Phenylstearic acid | 0.031 | 7.0% |
| 8-Phenylstearic acid | 0.04 | 9.0% |
| 9-Phenylstearic acid | 0.084 | 19.0% |
| 10-Phenylstearic acid | 0.079 | 17.8% |
| 11-Phenylstearic acid | 0.053 | 12.0% |
| 12-Phenylstearic acid | 0.049 | 11.1% |
| 13-Phenylstearic acid | 0.04 | 9.0% |
| 14-Phenylstearic acid | 0.018 | 4.1% |
| 15-Phenylstearic acid | 0.009 | 2.0% |
| 16-Phenylstearic acid | 0.018 | 4.1% |
| 17-Phenylstearic acid | 0.022 | 5.0% |

### Deoxygenation (Hydrogenation)

The accumulated stearic acid (100 grams) obtained from the Phenylation example was then charged to a second batch reactor having 1.5 liters volume along with five grams of a palladium on carbon (Pd/C) powdered catalyst, and 0.3 liters of n-octane solvent. Specifically, the Pd/C catalyst contained 5 wt. % palladium on carbon support and was pre-activated by hydrogen gas. Nitrogen gas was then injected to the batch reactor at about 0.5 L/min for 30 minutes at room temperature concurrently with stirring at 100 rpm. Hydrogen gas then charged to the reactor vessel at about 25 bar gauge, with the reactor subsequently heated to approximately 350° C. at a ramp of 10° C./min. Temperature was then maintained for 5 hours. The reactor was then cooled and depressurized to ambient conditions. Subsequently, the catalyst particles and n-octane solvent were removed by filtration and distillation respectively. The obtained product was observed to contain alkylaromatics, specifically about 90 mol. % phenylheptadecane. The phenylheptadecane was also generally observed to have a phenyl ring at the 6-16 position. Total deoxygenation/decarboxylation of the sample was approximately 95%, measured as

$$1 - ( \frac{carboxylic acid in product}{carboxylic acid in feed} ) .$$

Similar to the Phenylation example, the deoxygenation example was repeated to accumulate phenylheptadecane for the subsequent reactions.

### Supercritical Water Conversion

The hydrogenated phenylheptadecane product (2.1 grams) along with 15 mL of deionized water was then added to another batch reactor (0.1 liters). After purging with nitrogen ten times, the reactor was closed. Nitrogen was then again injected to reach a reactor pressure of approximately 0.2 MPa gauge. The reactor was then heated to approximately 425° C. with 27 MPa gauge recorded for internal pressure. The temperature was then held for about 20 minutes, with the reactor vessel subsequently being quenched in a water bath before being depressurized. The organic content was then recovered from the reactor, upon which the LAO were isolated to approximately 1.1 grams. The distribution of the LAO's is shown below in Table 2.

TABLE 2
LAO Product Distribution

| LAO Component | Weight % |
|---|---|
| 1-octene | 18.0% |
| 1-nonene | 20.0% |
| 1-decene | 13.0% |
| 1-undecene | 12.0% |
| 1-dodecene | 6.0% |
| % C₈-C₁₂ LAOs in LAO Product | 69.0% |
| % Non C₈-C₁₂ LAOs in LAO Product | 31.0% |
| Total LAO Yield (based on alkylaromic feed) | 52.4% |
| C₈-C₁₂ LAO Yield (based on alkylaromatic feed) | 36.1% |

Without being limited by theory, it may be expected that yields of almost entirely LAOs would not be expected from the feedstock due to one of the primary products of the initial beta scission being a phenyl group, as shown in FIG. 1 (the phenyl group of which predominately converts to toluene). Particularly, as shown in Table 3 below, the amount of phenyl groups present in the supercritical water product are estimated around 24 wt. %, whereas the alkyl groups, (groups susceptible to conversion to LAOs) number about 76 wt. %. In the 2.1 gram product of the SCW reactor, LAO product numbers about 52.4 wt. %, whereas Non-LAOs (toluene, unreacted phenylated alkyls, etc.) number about 47.6 wt. %.

Accordingly, about 70% of the total alkyl chains were converted into LAOs. However, given that in the ideal reaction pathway a portion of the alkyl chain is sacrificed to the phenyl group to form toluene, effective LAO yield is actually higher as the amount of total alkyl chain eligible for LAO conversion amounts to 1.49 grams instead of 1.6 grams. Accordingly, effective LAO yield and effective C8-C12 LAO yield amount to 73.8% and 50.9% respectively, which represents a large yield of LAO product and particularly preferred LAO products from the feed.

TABLE 3
Effective LAO Yield

| | | | |
|---|---|---|---|
| | Mol. | Wt. | |
| Formula | Weight | (grams) | Wt % |

Input to SCW

| | | | | |
|---|---|---|---|---|
| Phenylheptadecane | C23H40 | 316 | 2.1 | 100.0% |
| Phenyl group | C6H5 | 77 | 0.5 | 24.4% |
| Alkyl group | C17H35 | 239 | 1.6 | 75.6% |

Output from SCW

| | | |
|---|---|---|
| LAOs | 1.1 | 52.4% |
| Non-LAOs | 1.0 | 47.6% |

Maximum LAO Yield Accounting for Toluene

| | | | | |
|---|---|---|---|---|
| Toluene | C7H8 | 92 | 0.61 | 29% |
| Effective Maximum LAO Yield | | | 1.49 | 71% |

Effective LAO Yield

| | | |
|---|---|---|
| LAO Output | 1.1 | 52.4% |
| Effective Maximum LAO Yield | 1.49 | 71% |
| Effective LAO Yield | 73.8% | 73.8% |
| % C₈-C₁₂ LAOs in LAO | 69.0% | 69.0% |
| Product | | |
| Effective C8-C12 LAO Yield | 50.9% | 50.9% |

Consequently, the embodiments herein show potential for selectively converting a fatty acid and aromatic feed to linear alpha olefins, and particularly for selective conversion to C₈-C₁₂ linear alpha olefins.

It is noted that recitations in the present disclosure of a component of the present disclosure being “configured”, “operable”, or “sufficient” in a particular way, to embody a particular property, or to function in a particular manner, are structural recitations, as opposed to recitations of intended use. More specifically, the references in the present disclosure to the manner in which a component is “configured, “operable”, or “sufficient” denotes an existing physical condition of the component and, as such, is to be taken as a definite recitation of the structural characteristics of the component.

It is also noted that recitations herein of “at least one” component, element, etc., should not be used to create an inference that the alternative use of the articles “a” or “an” should be limited to a single component, element, etc. The singular forms “a,” “an” and “the” include plural referents, unless the context clearly dictates otherwise.

Throughout this disclosure ranges are provided. It is envisioned that each discrete value encompassed by the ranges are also included. Additionally, the ranges which may be formed by each discrete value encompassed by the explicitly disclosed ranges are equally envisioned.

It is noted that one or more of the following claims utilize the term “wherein” as a transitional phrase. For the purposes of defining the present invention, it is noted that this term is introduced in the claims as an open-ended transitional phrase that is used to introduce a recitation of a series of characteristics of the structure and should be interpreted in like manner as the more commonly used open-ended preamble term “comprising.” It is noted that the use of the terms “having” or “including”, or grammatical variations thereof, in this disclosure should also be interpreted in like manner as the more commonly used open-ended preamble term “comprising”.

As used in this disclosure, terms such as “first” and “second” are arbitrarily assigned and are merely intended to differentiate between two or more instances or components. It is to be understood that the words “first” and “second” serve no other purpose and are not part of the name or description of the component, nor do they necessarily define a relative location, position, or order of the component. Furthermore, it is to be understood that the mere use of the term “first” and “second” does not require that there be any “third” component, although that possibility is contemplated under the scope of the present disclosure.

It is noted that terms like “preferably,” “commonly,” and “typically,” when utilized herein, are not utilized to limit the scope of the claimed invention or to imply that certain features are critical, essential, or even important to the structure or function of the claimed invention. Rather, these terms are merely intended to identify particular aspects of an embodiment of the present disclosure or to emphasize alternative or additional features that may or may not be utilized in a particular embodiment of the present disclosure.

Having described the subject matter of the present embodiments herein in detail and by reference to specific embodiments thereof, it is noted that the various details disclosed herein should not be taken to imply that these details relate to elements that are essential components of the various embodiments described herein, even in cases where a particular element is illustrated in each of the drawings that accompany the present description. Further, it will be apparent that modifications and variations are possible without departing from the scope of the present embodiments including, but not limited to, embodiments defined in the appended claims. More specifically, although some aspects of the present embodiments are identified herein as preferred or particularly advantageous, it is contemplated that the present embodiments is not necessarily limited to these aspects.

1. A process for producing linear alpha olefins utilizing a supercritical water unit, the process comprising:
phenylating a free fatty acid stream with an aromatics stream in the presence of a Lewis Acid catalyst, thereby forming a phenylated free fatty acid stream;
deoxygenating the phenylated free fatty acid stream to form an alkylaromatic stream;
introducing water and the alkylaromatic stream to a supercritical water unit, wherein the water is either at supercritical conditions prior to entering the supercritical water unit or converted to supercritical conditions within the supercritical water unit; and
converting the alkylaromatic stream with the water within the supercritical water unit, thereby forming a linear alpha olefin-containing product.

2. The process of claim 1, wherein deoxygenating the phenylated free fatty acid stream to form the alkylaromatic stream comprises:
thermally cracking the phenylated free fatty acid stream in the presence of steam or water to form the alkylaromatic stream;
catalytically cracking the phenylated free fatty acid stream in the presence of a catalyst as well as steam or water to form the alkylaromatic stream; or
hydrogenating the phenylated free fatty acid stream in the presence of hydrogen to form the alkylaromatic stream.

3. The process of claim 2, wherein the phenylated free fatty acid stream is deoxygenated in the presence of an n-alkane solvent, each of the n-alkanes of the n-alkane solvent having greater than or equal to six carbons.

4. The process of claim 2, wherein the phenylated free fatty acid stream is hydrogenated in the additional presence of a noble metal catalyst, a transition metal sulfide catalyst, or combinations thereof.

5. The process of claim 1, wherein:
the free fatty acids comprise palmitic acid, stearic acid, oleic acid, linoleic acid, arachidic acid, eicosenoic acid, behenic acid, or combinations thereof; and
the aromatics stream comprises benzene, toluene, xylene, ethylbenzene, diethylbenzene, or combinations thereof.

6. The process of claim 1, further comprising separating a non-phenylated portion of the phenylated free fatty acid stream prior to deoxygenating the phenylated free fatty acid stream.

7. The process of claim 1, wherein the free fatty acid stream is derived from triglycerides.

8. The process of claim 7, further comprising:
hydrolyzing the triglycerides in the presence of a second water stream to form the free fatty acid stream and glycerol; and
separating the free fatty acid stream from the glycerol.

9. The process of claim 1, further comprising:
introducing the linear alpha olefin-containing product to a gas-liquid separator to form a gas product stream and a liquid product stream;
introducing the gas product stream to a first oil-water separator to form a gas hydrocarbon product, a liquid hydrocarbon product, and a residue water stream; and
introducing the liquid product stream to a second oil-water separator to form additional gas hydrocarbon product, additional liquid hydrocarbon product, and additional residue water stream.

10. The process of claim 9, further comprising introducing the liquid hydrocarbon product to a fractionation unit, thereby forming a linear alpha olefin fraction and optionally:
a BTX fraction;
an alkykated benzene fraction;
an unreacted alkylaromatic fraction; or
combinations thereof.

11. The process of claim 10, further comprising:
quenching the linear alpha olefin-containing product prior to sending the linear alpha olefin-containing product to the gas-liquid separator;
depressurizing the linear alpha olefin-containing product prior to sending the linear alpha olefin-containing product to the gas-liquid separator; or
both.

12. The process of claim 11, wherein:
the linear alpha olefin-containing product is quenched to a temperature of from 20° C. to 250° C.; and
the linear alpha olefin-containing product is depressurized to a pressure of from 0.1 megapascals (MPa) to 5 MPa.

13. The process of claim 1, wherein:
the water is preheated to a temperature of from 374° C. to 800° C. prior to being introduced to the supercritical water unit;
the alkylaromatic stream is preheated to a temperature of from 50° C. to 250° C. prior to being introduced to the supercritical water unit; or
both.

14. The process of claim 1, further comprising:
mixing the water and the hydrocarbon-containing stream prior to the water and the hydrocarbon-containing stream being introduced to the supercritical water unit; and
preheating the water-hydrocarbon-containing stream mixture to a temperature of from 380° C. to 450° C. prior to introducing the water-hydrocarbon-containing stream mixture to the supercritical water unit.

15. The process of claim 1, wherein an alkylaromatic stream to water ratio introduced to the supercritical water unit is from 10:1 to 1:10 alkylaromatic stream to water.

16. The process of claim 4, wherein the hydrogenation occurs at:
a temperature of greater than 250° C.;
a pressure of greater than 0.5 MPa;
a liquid hourly space velocity of greater than 0.1 hr⁻¹; or
combinations thereof.

17. The process of claim 2, wherein the hydrogenation occurs at:
a temperature of greater than 300° C.;
a pressure of greater than 0.1 MPa;
a residence time of greater than 1 minute; or
combinations thereof.

18. The process of claim 1, wherein the phenylation occurs at:
a ratio of the free fatty acid to the aromatics stream is from 1:2 to 1:100;
an operating temperature of from 10° C. to 500° C.;
an operating pressure of from 0.1 megapascals (MPa) to 15 MPa;
a weight hourly space velocity (WHSV) of from 0.05 hr⁻¹ to 100 hr⁻¹; or
combinations thereof.

19. The process of claim 1, wherein the supercritical water unit operates:
at a temperature of from 380° C. to 450° C.;
with a residence time of from 10 seconds to 60 minutes;
in an upflow or downflow configuration; or
combinations thereof.

20. A system for producing linear alpha olefins, the system comprising:
a pretreatment unit comprising a phenylation unit and a deoxygenation unit, wherein:
the phenylation unit is configured to receive a free fatty acid stream and an aromatics stream, and
the deoxygenation unit is fluidly connected downstream from the phenylation unit and configured to receive at least phenylated free fatty acid stream;
a supercritical water unit fluidly connected downstream from the deoxygenation unit of the pretreatment unit, wherein the he supercritical water unit is configured to:
receive an alkylaromatic stream from the deoxygenation unit and water,
operate under supercritical water conditions, and
produce a linear alpha olefin-containing product.
